(19) United States
(12) Patent Application Publication
(10) Pub. No.: US 2010/0190873 A1
(43) Pub. Date: Jul. 29, 2010

(54) Extract of the plant Ashwaganda (Withania Somnifera) consisting of the seeds of said plant dried and processed into a flour to be used as an ingredient to be incorporated in tablets and/or capsules for human consumption and/or to be available as a base for solvent extraction where said extaction(s) would be used for tablets and/or capsules for human consumption and/or human skin creams and/or lotions

(76) Inventor: Robert D. Bibb, Myrtle Beach, SC (US)

Correspondence Address: Robert D. Bibb, 7554 Siena Blvd., Myrtle Beach, SC, 29572, US

(21) Appl. No.: 12/321,731

(22) Filed: Jan. 26, 2009

(51) Int. Cl. A61K 47/06; A21D 2/00

(52) U.S. Cl. 514/783; 426/622

(57) ABSTRACT

This patent presents a previously undescribed flour obtained from the seeds of Ashwaganda (Withania Somnifera) to be incorporated into tablets and/or capsules or act as a base for solvent extraction where said extractions will be incorporated into tablets and/or capsules and where said extractions may be incorporated into creams/and/or lotions for application to human skin.

### FIELD OF THE INVENTION

The present invention relates generally to a previously undescribed use of a particular portion of the plant known as Ashwaganda (Withania Somnifera) for incorporation into tablets and/or capsules for human consumption and solvent extraction(s) of said plant portion to be used for human consumption and/or application to human skin and/or be incorporated into tablets and/or capsules for human consumption.

It can generally be appreciated that whole leaf and root portions of the plant have been used in Ayurevedic medicine for over 5000 years. The plant also belongs to a category of plants generally regarded as safe (GRAS).¹ ¹ Deocris C. et al.: Merger of Ayurvedic and Tissue Culture-Based Functional Genomics: Inspirations from Systems Biology. Journal Of Translational Medicine. March 18, 2008: 6:14.

Numerous studies have investigated the beneficial effects of the roots and leaves and extracts of these portions of the plant.^{2 3 4 5} but there has been no reported use of the seed extract(s) of the Ashwaganda (Withania Somnifera) plants. ² Khanna D. et al. Natural Products as a gold mine for arthritis treatment. Cur. Opi. Pharmacol. June 2007;7(3):344-51.³ Widodo N. et al. Selective Killing of Cancer Cells by Leaf Extract of Ashwaganda: Identification of a Tumor-Inhibitory Factor and the First Molecular Insights to its Effect. Clinical Cancer Research. Apr. 1, 2007;(13): 2298-2306.⁴ Kumar A. et al. Protective effect of Withania Dunal on the behavioral and biochemical alterations in sleep-disturbed mice (Grid over water suspended method). Indian J. Exp Biol. June 2007;45(6):524-8.⁵ Sumantran V N. Et al. Chondroprotective potential of root extracts of Withania Somnifera in osteoarthritis. J. Biosci March 2007;32(2):299=307.

It can also generally be appreciated that the seeds of most plants store the bioactive compounds found in the pulp, roots and leaves in quantities sufficient to make them a candidate for use as active forms of the plant. Examples of such seed benefits include extracts of cranberry seeds, grape seeds, soy bean seeds and safflower seeds.^{6 7 8} The difficulty with the use of the whole seed is the seed coat. This coat prevents access to the active seed contents. Techniques to extract the content of the seeds have been perfected and involve drying the seeds in a rotating mesh drum at elevated temperatures or freeze drying the seed. With either technique the seeds must be ground into a flour by any one of several commercial methods to allow bioavailability of the bioactive compounds. Bioavaiability of certain compounds is then either obtained through absorption into the blood stream through human ingestion or the internal contents of the seed can be released from inside the seed coats making them available for solvent extraction. ⁶ Chatelain K. et al. Cranberry and Grape Seed Extracts Inhibit the Proliferative Phenotype of Oral Squamous Cell Carcinoms. Annals of Oncology. Published on-line Jul. 23, 2008.⁷ Wei M. et al. Soybean seed extracts preferentially express genomic loci of Bradyrhizobium japonicum in the initial interaction with soybean. Glycine max(L.) Merr. DNA Res. August 2008;15(4):201-14.⁸ Moon K. et al. Safflower seed extract lowers plasma and hepatic lipids in rats fed high-cholesterol diet. Nutrition Research June 2001;21(6): 895-904.

### SUMMARY OF THE PRESENT INVENTION

The present invention takes advantage of preexisting techniques to create a previously undescribed Ashwaganda (Withania Somnifera) seed flour for use as a portion of the Ashwaganda (Withania Somnifera) plant. Said flour will be used for incorporation into tablets and/or capsules. Extracts of created seed flour will be incorporated into creams, lotions, tablets and/or capsules for either human consumption or for application to human skin.

### DETAILED DESCRIPTION OF THE PREFERRED EMBODIMENTS

The present invention is a newly described flour obtained from the seed of the plant Ashwaganda (Withania Somnifera) and the uses of this flour in tablets and/or capsules for human consumption and the extracts; thereof, for use in creams and/or lotion for application to human skin or incorporation into tablets and/or capsules for human consumption. With respect to the above descriptions, it is realized that the optimum quantities of the seed flour and/or extracts to be incorporated into capsules, tablets, creams or lotions will vary and; therefore, the foregoing description should be illustrative of the principles of the invention. Further, since numerous modifications and changes will occur in the exact composition of the tablets, capsules, creams and lotions, all suitable modifications and equivalents that may be resorted to should be included in the scope of this invention.

What is claimed is:

1. That I have invented a previously undescribed flour from the seeds of the plant Ashwaganda (Withania Somnifera).

2. That said seed flour will be incorporated into tablets and/or capsules for human consumption.

3. That said seed flour will be used as a base to extract bioactive compounds to be incorporated into creams and/or lotions for application to human skin.
